(19) United States
(12) Patent Application Publication
(10) Pub. No.: US 2009/0214458 A1
(43) Pub. Date: Aug. 27, 2009

(54) COSMETIC COMPOSITION COMPRISING AT LEAST ONE SPECIFIC SILICONE COPOLYMER, AT LEAST ONE VOLATILE SOLVENT AND AT LEAST ONE CERTAIN SILICONE RESIN

(76) Inventors: Gaelle BRUN, Paris (FR); Arnaud Bonnamy, Versailles (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/392,549

(22) Filed: Feb. 25, 2009

Related U.S. Application Data
(60) Provisional application No. 61/064,417, filed on Mar. 5, 2008.

(30) Foreign Application Priority Data
Feb. 26, 2008 (FR) 0851215

(51) Int. Cl. A61K 8/58; A61Q 5/12; A61Q 5/00

(52) U.S. Cl. 424/70.6; 424/70.12

(57) ABSTRACT

The present disclosure relates to a keratin fiber treatment composition comprising at least one copolymer comprising at least one silicone resin and at least one fluid silicone, at least one volatile solvent, and at least one silicone resin having at least one trifunctional unit of the formula (R)SiO_{3/2} (T unit). Coatings are thus obtained on the keratin fibers that give the hair shampoo-remanent volume, mass and body, while at the same time maintaining the physical qualities of the keratin fiber.

This application claims benefit of U.S. Provisional Application No. 61/064,417, filed Mar. 5, 2008, the contents of which are incorporated herein by reference. This application also claims benefit of priority under 35 U.S.C. § 119 to French Patent Application No. FR 0851215, filed Feb. 26, 2008, the contents of which are also incorporated herein by reference.

The present disclosure relates to a composition for treating keratin fibers, including but not limited to human keratin fibers, for example, the hair, and also to a process for treating keratin fibers using this composition.

Hair can generally be damaged and embrittled by the action of external atmospheric agents such as light and bad weather, and by mechanical or chemical treatments such as brushing, combing, bleaching, permanent waving and/or dyeing. As a result, the hair is often difficult to manage, and in particular can be difficult to disentangle or style, and a head of hair, even a rich head of hair, can have difficulty in maintaining an attractive style due to the fact that the hair lacks vigour, volume and liveliness.

This degradation of the hair is moreover increased by repetition of the permanent dyeing treatments of the hair, which can comprise applying to the hair one or more dye precursors and an oxidizing agent.

Thus, to overcome this, it is now common practice to use styling products that allow hair to be conditioned, for example to give it body, mass or volume.

These styling products are generally cosmetic hair compositions comprising one or more polymers that have high affinity for the hair and that usually have the function of forming a film at its surface in order to modify its surface properties, such as to condition it or give it certain optical properties.

One drawback associated with the use of these hair compositions lies in the fact that the cosmetic effects imparted by such compositions have a tendency to disappear, sometimes from the very first shampoo wash. This can be true when one of the effects imparted is a color effect provided by pigments.

In order to overcome this drawback, it may be envisaged to increase the remanence of the polymer deposit by directly performing a group polymerization of certain monomers on the hair. However, the treatments thus obtained can lead to degradation of the hair fiber and hair thus treated is generally difficult to disentangle.

It is moreover known practice to coat the hair with a composition comprising an electrophilic monomer of cyanoacrylate type for example as discussed in patent application FR 2 833 489. Such a composition may afford perfectly coated and non-greasy hair. However, the coating obtained can require certain operating conditions due to the reactivity of the electrophilic monomer. Moreover, the coating obtained with these electrophilic monomers can become tacky with fatty substances such as sebum.

Certain silicone copolymers comprising a silicone resin segment and a fluid silicone segment also exist, and are more commonly known as BioPSA. These copolymers are described in, for example, patent applications WO 03/026 596, WO 2004/073 626, WO 2007/051 505 and WO 2007/051 506; these applications also describe various cosmetic applications such as application to the hair, the nails and the skin. The feel obtained with these copolymers is generally tacky.

The present disclosure provides an easy-to-use process for treating keratin fibers, including, for example, human hair, which can produce coatings that can have at least one property of being remanent with respect to shampooing and to the various attacking factors to which the hair may be subjected, such as blow-drying and perspiration, while at the same time being capable of showing better tolerance to fatty substances such as sebum and not developing a tacky nature. In some embodiments, the present disclosure provides processes for obtaining easy-to-use colored, non-tacky coatings, which can be resistant to external agents and which can respect the integrity of keratin fibers.

One aspect of the present disclosure is a keratin fiber treatment composition comprising at least one copolymer comprising at least one silicone resin and at least one fluid silicone; at least one volatile solvent; and at least one silicone resin comprising at least one trifunctional unit of formula (R)SiO_{3/2}, wherein R is chosen, independently by trifunctional unit, from hydrocarbon-based groups and hydroxyl groups, on the condition that at least one R is a hydrocarbon-based group.

As used herein, “hydrocarbon-based” means that a group, molecule, or ion comprises primarily carbon and hydrogen atoms.

Another aspect of the present disclosure is a process for treating keratin fibers comprising the application of the compositions as disclosed herein.

Still another aspect of the present disclosure is a process of obtaining a colored coating on hair comprising application of the compositions as disclosed herein, wherein the compositions comprise at least one pigment.

A further aspect of the present disclosure is a method for preparing compositions for treating hair as disclosed herein.

These coatings can make it possible to give the hair shampoo-remanent volume, mass and body, while at the same time maintaining the physical qualities of the keratin fiber, with, in addition, a remanent color effect when the composition contains pigments. Such a coating can be, for example, resistant to the external attacking factors to which the hair may be subjected, such as blow-drying and perspiration. It furthermore can afford permanent colorations without using oxidizing agents liable to degrade the hair.

The coating thus formed can be in the form of a smooth, uniform deposit and can show excellent adhesion to the hair. Moreover, it has been found that the hairs can remain individualized and can be styled without any problem, and that the styling properties afforded to the fiber can be shampoo-remanent. Moreover, the feel obtained may have minimal heaviness and can be non-tacky.

Hereinafter, unless indicated otherwise, all ranges described are inclusive.

### Copolymer Based on Silicone Resin and on Fluid Silicone

The silicone copolymer defined according to the present disclosure is derived from the reaction between at least one silicone resin and at least one fluid silicone.

Such copolymers are described, for example, in “Silicone Pressure Sensitive Adhesive”, Sobieski and Tangney, Handbook of Pressure Sensitive Adhesive Technology (D. Satas Ed.), Von Nostrand Reinhold, New York.

In some embodiments, the at least one silicone resin is present in the copolymer in a total content ranging from 45% to 75% (relative to the total mass of silicone) and the at least one fluid silicone is present in a total content ranging from 25% to 55%, the sum of the percentages of silicone resin and of fluid silicone being equal to 100. In some embodiments, the at least one silicone resin is present in a total content ranging from 55% to 65% (relative to the total mass of silicone) and the at least one fluid silicone is present in a total content ranging from 35% to 45%, the sum of the percentages of silicone resin and of fluid silicone being equal to 100.

In some embodiments, the at least one silicone resin is the product of condensation of SiO₂ groups and of R₃(SiO)_{1/2} (triorganosilyl) groups for which each R group is independently selected from methyl, ethyl, propyl and vinyl groups and for which the ratio between the SiO₂ functions and the R₃(SiO)_{1/2} functions of the silicone resin ranges from 0.6 to 0.9. Triorganosilyl groups that may be used to form the silicone resin may be trimethylsilyl, triethylsilyl, methylmethylpropylsilyl or dimethylvinylsilyl groups, and mixtures thereof. For example, in at least one embodiment of the present disclosure, the trimethylsilyl group is used.

In some embodiments, the fluid silicone is a diorganopolysiloxane bearing OH end functions, having a viscosity ranging from 100 and 100,000 cSt at 25° C., for which the substituents of the diorganopolysiloxane are independently chosen from methyl, ethyl, propyl and vinyl groups. The diorganopolysiloxanes may be linear polymers. Examples of diorganopolysiloxanes may be, in a non-limiting manner, a polydimethylsiloxane, an ethylmethyl polysiloxane, a copolymer of dimethylsiloxane and of methylvinylsiloxane, and mixtures of such polymers or copolymers containing OH end groups. The diorganopolysiloxane may be a polydimethylsiloxane.

Examples of synthesis of such a copolymer are described, for example, in U.S. Pat. No. 5,162,410 or in patent CA 711 756.

The copolymers according to the present disclosure may thus be prepared by heating the following mixture:

from 45% to 75% by mass of silicone resin, being the product of condensation of SiO₂ and R₃(SiO)_{1/2} units for which each R group is independently selected from methyl, ethyl, propyl and vinyl groups and for which the ratio between the SiO₂ functions and the R₃(SiO)_{1/2} functions of the silicone resin ranges from 0.6 to 0.9;

from 25% to 55% by mass of fluid diorganopolysiloxane containing OH end functions, with a viscosity ranging from 100 and 100,000 cSt at 25° C., for which the substituents of the diorganopolysiloxane are independently chosen from methyl, ethyl, propyl and vinyl groups;

from 0.001% to 5% of a suitable catalyst, which may be an organic aliphatic amine compound, a primary amine, secondary amine, tertiary amine, a carboxylic acid salt of the amines mentioned above, or a and quaternary ammonium salt.

The mixture is heated to a temperature ranging from 80° C. and 160° C. until the adhesive nature of the resulting silicone copolymer is obtained.

In some embodiments, the copolymers are those sold by Dow Corning under the reference BIO-PSA®, these BIO-PSA® copolymers possibly being in two forms, standard or amine-compatible, and being provided in different solvents with several silicone resin/fluid silicone ratios. Mention may be made of the grades 7-4400, 7-4500 and 7-4600.

In some embodiments, the quantity of copolymer is greater than 1% by weight of the total weight of the composition.

The copolymer may be present in the composition in a content of greater than 1% and up to 40% by weight, or it may range from 1.5% to 20% by weight or from 1.5% to 15% by weight, relative to the total weight of the composition.

### Volatile Solvent

The composition contains at least one volatile solvent.

As used herein, the term “volatile solvent” means a compound that is liquid at room temperature (20° C.) and at atmospheric pressure (760 mmHg), with a vapour pressure at 20° C. of greater than 0.1 mmHg. In some embodiments, the vapour pressure at 20° C. of the volatile solvent may range from 0.1 to 300 mmHg, or from 0.5 to 200 mmHg.

The at least one volatile solvent may be chosen from non-silicone organic solvents and silicon organic solvents. Volatile non-silicone organic solvents that may be mentioned include:

volatile C₁-C₄ alkanols such as ethanol or isopropanol;

volatile C₅-C₇ alkanes such as n-pentane, hexane, cyclopentane, 2,3-dimethylbutane, 2,2-dimethylbutane, 2-methylpentane or 3-methylpentane;

esters of liquid C₁-C₂₀ acids and of volatile C₁-C₈ alcohols such as methyl acetate, n-butyl acetate, ethyl acetate, propyl acetate, isopentyl acetate or ethyl 3-ethoxypropionate;

ketones that are liquid at room temperature and volatile, such as methyl ethyl ketone, methyl isobutyl ketone, diisobutyl ketone, isophorone, cyclohexanone or acetone;

volatile polyols such as propylene glycol;

volatile ethers such as dimethoxymethane, diethoxyethane or diethyl ether;

volatile glycol ethers such as 2-butoxyethanol, butyl diglycol, diethylene glycol monomethyl ether, propylene glycol n-butyl ether or propylene glycol monomethyl ether acetate;

volatile hydrocarbon-based oils such as, for example, volatile hydrocarbon-based oils containing from 8 to 16 carbon atoms, and mixtures thereof, and branched C₈-C₁₆ alkanes, for instance C₈-C₁₆ isoalkanes (also known as isoparaffins), isododecane, isodecane and, for example, the oils sold under the trade names Isopar or Permethyl, and mixtures thereof. Mention may also be made of isohexyl or isodecyl neopentanoate;

volatile C₄-C₁₀ perfluoroalkanes such as dodecafluoropentane, tetradecafluorohexane or decafluoropentane;

volatile perfluorocycloalkyls such as perfluoromethylcyclopentane, 1,3-perfluorodimethylcyclohexane and perfluorodecalin, sold, respectively, under the names Flutec PC1®, Flutec PC3® and Flutec PC6® by the company F2 Chemicals, and also perfluorodimethylcyclobutane and perfluoromorpholine;

the volatile fluoroalkyl or heterofluoroalkyl compounds corresponding to the following formula:

CH₃—(CH₂)ₙ[Z]ₜ-X—CF₃

in which t is 0 or 1; n is 0, 1, 2 or 3; X is a linear or branched divalent perfluoroalkyl group containing from 2 to 5 carbon atoms, and Z represents O, S or NR, R being a hydrogen atom or a group —(CH₂)ₙ—CH₃ or a group —(CF₂)ₘ—CF₃, m being 2, 3, 4 or 5.

Among the volatile fluoroalkyl or heterofluoroalkyl compounds that may be mentioned are methoxynonafluorobutane sold under the name MSX 4518® and HFE-7100® by the company 3M, and ethoxynonafluorobutane sold under the name HFE-72000 by the company 3M.

In some embodiments, the solvent has a boiling point less than 200° C.

In at least one embodiment, the at least one non-silicone organic solvent is chosen from ethanol, isopropanol, acetone, and alkanes which are liquid at 25° C. and at atmospheric pressure (760 mmHg) such as isododecane.

Volatile silicone compounds that may be mentioned include low-viscosity silicon compounds chosen from linear or cyclic silicones containing from 2 to 7 silicon atoms, these silicones optionally comprising alkyl or alkoxy groups containing from 1 to 10 carbon atoms, for example octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane and decamethyltetrasiloxane, and mixtures thereof. According to at least one embodiment, the silicone compound is chosen from cyclopentadimethylsiloxane and dodecamethylcyclohexasiloxane.

According to at least one embodiment, the at least one volatile silicone solvent has a viscosity of less than 50 centistokes.

In some embodiments, the volatile silicone is chosen from decamethylcyclopentasiloxane, dodecamethylcyclohexasiloxane, octamethyltrisiloxane and decamethyltetrasiloxane.

Examples that may be mentioned include the decamethylcyclopentasiloxane sold under the name DC-245 by the company Dow Corning, dodecamethylcyclo-hexasiloxane sold under the name DC-246 by the company Dow Corning the octamethyltrisiloxane sold under the name DC-200 Fluid 1 cSt by the company Dow Corning, and the decamethyltetrasiloxane sold under the name DC-200 Fluid 1.5 cSt by the company Dow Corning.

This volatile silicone generally has a low viscosity, for example a viscosity of less than 10 cSt at 25° C.

In some embodiments, the volatile silicone is cyclic, for example, the decamethylcyclopentasiloxane sold under the name DC-245 by the company Dow Corning or dodecamethylcyclohexasiloxane sold under the name DC-246 by the company Dow Corning.

The at least one volatile solvent may be present in the composition in a content ranging from 0.1% to 95% by weight, from 1% to 70% by weight, or from 5% to 90% by weight, relative to the total weight of the composition.

### Silicone Resin Comprising at Least One T Unit

As used herein, the term “resin” means a crosslinked or non-crosslinked three-dimensional structure. Examples of polysiloxane T resinshat may be mentioned include silsesquioxanes and siloxysilicates.

The nomenclature of silicone resins is known under the name MDTQ, the resin being described as a function of the various siloxane monomer units it comprises, each of the letters MDTQ characterizing a type of unit.

The letter M represents the monofunctional unit, for example, of formula (CH₃)₃SiO_{1/2}, the silicon atom being connected to only one oxygen atom in the polymer comprising this unit.

The letter D means a difunctional unit, for example, (CH₃)₂SiO_{2/2} in which the silicon atom is connected to two oxygen atoms.

The letter T represents a trifunctional unit, for example, of formula (CH₃)SiO_{3/2}.

In the M, D and T units listed as examples above, at least one of the methyl groups may be substituted. In some embodiments, the at least one silicone resin comprising at least one trifunctional unit of formula (R)SiO_{3/2} is chosen from the silsesquioxanes of formula: ((R′)SiO_{3/2})ₓ, in which x ranges from 100 to 500 and R′ is chosen, independently by trifunctional unit, from a hydrocarbon-based group containing from 1 to 10 carbon atoms or a hydroxyl group, on the condition that at least one R′ is a hydrocarbon-based group. In some embodiments, the hydrocarbon-based group containing from 1 to 10 carbon atoms is a methyl group. In some embodiments, the at least one silicone resin comprising at least one trifunctional unit of formula (R)SiO_{3/2} is chosen from the silsesquioxanes of the formula: ((R′)SiO_{3/2})ₓ, in which x ranges from 100 to 500 and R′ is chosen, independently by unit, from CH₃, a hydrocarbon-based group containing from 2 to 10 carbon atoms, or a hydroxyl group, on the condition that at least one R′ is a hydrocarbon-based group.

Finally, the letter Q means a tetrafunctional unit SiO_{4/2} in which the silicon atom is bonded to four hydrogen atoms, which are themselves bonded to the rest of the polymer.

Various resins with different properties may be obtained from these different units, the properties of these polymers varying as a function of the type of monomers (or units), of the type and number of substituted groups, of the length of the polymer chain, of the degree of branching and of the size of the side chains.

The silicone resin contains at least one T unit. It may thus be, for example, a T, MT, MTQ or MDTQ resin.

In some embodiments, the unit composition of the silicone resin is at least 50% T units, or at least 80% T units.

In some embodiments, the T resins may contain M, D and Q units such that at least 80 mol % or at least 90 mol %, relative to the total amount of silicones, are T units. The T resins may also contain hydroxyl and/or alkoxy groups. The T resins may have a total weight of hydroxyl functions ranging from 2% to 10% and a total weight of alkoxy functions that may be up to 20%; in some embodiments, the total weight of hydroxyl functions ranges from 4% to 8% and the total weight of alkoxy functions may be up to 10%.

The silicone resin may be chosen from silsesquioxanes that are represented by the following formula: ((CH₃)SiO_{3/2})ₓ, in which x may be up to several thousand and the CH₃ group may be replaced with an R group, as described previously in the definition of the T units. The number x of T units of the silsesquioxane may be less than or equal to 500, or it may range from 50 to 500. The molecular weight of the silicone resin may range from 500 to 50,000 g/mol, from 500 to 20,000 g/mol, or from 500 to 10,000 g/mol.

The silicone resin may be film-forming. Specifically, not all silsesquioxanes are film-forming: for example, highly polymerized polymethylsilsesquioxanes such as Tospearl™ from Toshiba or KMP590 from Shin-Etsu are insoluble and are not film-forming. The molecular weight of these polymethylsilsesquioxanes is difficult to determine, but there are generally more than 1000 T units.

As examples of these silicone resins containing at least one T unit, mention may be made of:

polysilsesquioxanes of formula ((R)SiO_{3/2})ₓ (T units) in which x is greater than 100, in which the R groups may independently be methyl or other substituents as defined above;

polymethylsilsesquioxanes, which are polysilsesquioxanes in which R is a methyl group. Such polymethylsilsesquioxanes are described, for example, in U.S. Pat. No. 5,246,694;

polypropylsilsesquioxanes, in which R is a propyl group. These compounds and their synthesis are described, for example, in patent application WO 2005/075567;

polyphenylsilsesquioxanes, in which R is a phenyl group. These compounds and their synthesis are described, for example, in patent application US 2004/0180011.

Examples of commercially available polymethylsilsesquioxane resins that may be mentioned include those sold:

by the company Wacker under the reference Resin MK such as Belsil PMS MK: polymer comprising CH₃SiO_{3/2} repeating units (T units), which may also comprise up to 1% by weight of (CH₃)₂SiO_{2/2} units (D units) and having an average molecular weight of about 10 000 g/mol. It is thought that the polymer is in a “cage” and “ladder” configuration as represented in the figures below. The average molecular weight of the units in “cage” configuration has been calculated as 536 g/mol. The majority of the polymer is in the “ladder” configuration with ethoxy groups at the ends. These ethoxy groups represent 4.5% by mass of the polymer. As these end groups can react with water, a small and variable amount of SiOH groups may also be present.

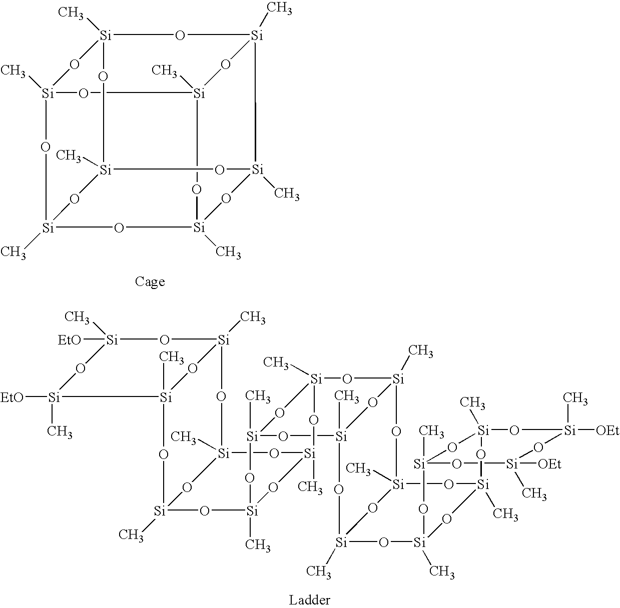

by the company Shin-Etsu under the references KR-220L, which are composed of T units of formula CH₃SiO_{3/2} and have Si—OH (silanol) end groups, under the reference KR-242A, which comprise 98% of T units and 2% of dimethyl D units and have Si—OH end groups or alternatively under the reference KR-251 comprising 88% of T units and 12% of dimethyl D units and have Si—OH end groups.

Examples of commercially available polypropylsilsesquioxane resins that may be mentioned include those sold:

by the company Dow Corning under the reference Dow Corning 670 Fluid, which is a polypropylsilsesquioxane diluted in D5.

Examples of commercially available polyphenylsilsesquioxane resins that may be mentioned include those sold:

by the company Dow Corning under the reference Dow Corning 217 Flake Resin, which is a polyphenylsilsesquioxane with silanol end groups;

by the company Wacker under the reference Belsil SPR 45 VP.

In some embodiments, the silicone resins are soluble or dispersible in the composition. The silicone resins may be soluble in volatile silicones and organic solvents. In some embodiments, the silicone resin is solid at 25° C.

The composition may comprise a plurality of types of silicone resins comprising at least one T unit. The at least one silicone resin comprising at least one T unit may be present in the composition in a concentration ranging from 0.1% to 20%, from 0.2% to 15%, or from 0.5% to 10%.

In some embodiments, the weight ratio of (1) the at least one silicone resin comprising the at least one trifunctional unit of formula (R)SiO_{3/2}, to (2) the at least one volatile solvent ranges from 1/200 to 1/10.

Non-Volatile Linear PDMS with a Viscosity of Greater than 5 cSt

In some embodiments, the composition comprises one or more linear non-volatile polydimethylsiloxanes (PDMSs) with a viscosity of greater than 5 cSt. This may improve the coating homogeneity.

The non-volatile linear PDMS with a viscosity of greater than 5 cSt may be a silicone gum or a silicone oil with a vapour pressure of less than 0.1 mmHg at 25° C.

The non-volatile linear PDMS with a viscosity of greater than 5 cSt may be chosen from polydimethylsiloxanes; alkyl dimethicones; polyphenylmethylsiloxanes such as phenyl dimethicones, phenyl trimethicones and vinyl methyl methicones; or silicones modified with at least one of aliphatic, aromatic, fluorinated aliphatic, fluorinated aromatic, hydroxyl, thiol, or amine groups.

The viscosity of the non-volatile linear PDMSs may be greater than 5 cSt at 25° C. In some embodiments, this viscosity ranges from 5 cSt at 25° C. to 5,000,000 cSt at 25° C., from 100 cSt at 25° C. to 4,000,000 cSt at 25° C., or from 5000 cSt at 25° C. to 4,000,000 cSt at 25° C.

The molecular weight may range from 500 to 800,000 g/mol, from 5,000 to 700,000 g/mol, or from 50,000 to 600,000 g/mol.

For example, this linear PDMS may be chosen from the silicones of formula (I):

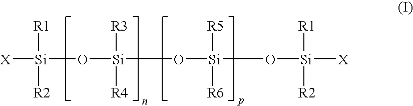

in which:

R₁, R₂, R₅ and R₆ are, together or separately, an alkyl group containing 1 to 6 carbon atoms;

R₃ and R₄ are, together or separately, an alkyl group containing from 1 to 6 carbon atoms, a vinyl group or an aryl group;

X is an alkyl group containing from 1 to 6 carbon atoms, a hydroxyl group, a vinyl group or an amine group;

n and p being integers chosen so as to have a compound with a viscosity of greater than 5 cSt; in some embodiments, the sum n+p is greater than 10.

Additional examples that may be mentioned include the following polydimethylsiloxanes:

the substituents R₁ to R₆ and X represent a methyl group, such as the product sold under the name Baysilicone TP 3898 by the company General Electric, and the product sold under the name AK 500 000 by the company Wacker;

the substituents R₁ to R₆ and X represent a methyl group, and p and n are such that the molecular weight is 120,000 g/mol, such as the product sold under the name Dow Corning 200 Fluid 60 000 CS by the company Dow Corning;

the substituents R₁ to R₆ and X represent a methyl group, and p and n are such that the molecular weight is 250,000 g/mol, for instance the product sold under the name Mirasil DM 500 000 by the company Rhodia and the product sold under the name Dow Corning 200 Fluid 500 000 cSt by the company Dow Corning;

the substituents R₁ to R₆ represent a methyl group, the group X represents a hydroxyl group, and n and p are such that the molecular weight of the polymer is 600,000 g/mol, for instance the product sold under the name SGM 36 by the company Dow Corning;

dimethicones of the (polydimethylsiloxane)(methylvinylsiloxane) type, such as SE63 sold by GE Bayer Silicones, and poly(dimethylsiloxane)(diphenyl) (methylvinylsiloxane) copolymers, and mixtures thereof.

The non-volatile linear PDMSs may be oxyalkylenated.

When they are present, the amount of linear non-volatile PDMSs may range from 0.1% to 20% or from 0.5% to 10%.

### Non-Volatile Organic Solvent

The composition may also comprise at least one non-volatile organic solvent.

Non-volatile organic solvents that may be mentioned include:

non-volatile aromatic alcohols such as benzyl alcohol or phenoxyethanol;

esters of liquid C₁-C₂₀ acids and of non-volatile C₁-C₈ alcohols, such as isopropyl myristate;

ethylene carbonate, propylene carbonate or butylene carbonate;

non-volatile polyols such as glycerol, ethylene glycol, dipropylene glycol or butylene glycol;

non-volatile glycol ethers, for instance diethylene glycol monomethyl ether or dipropylene glycol mono-n-butyl ether;

non-volatile hydrocarbon-based oils such as isohexadecane;

non-volatile liquid C₁₀-C₃₀ fatty alcohols such as oleyl alcohol; esters of liquid C₁₀-C₃₀ fatty alcohols such as benzoates of C₁₀-C₃₀ fatty alcohols and mixtures thereof; polybutene oil, isononyl isononanoate, isostearyl malate, pentaerythrityl tetraisostearate or tridecyl trimellitate;

non-volatile perfluoro solvents such as perfluoroperhydrophenanthrene, sold under the name Flutec PC11® by the company F2 Chemicals.

The non-volatile organic solvent(s) may be present in the composition in a content ranging from 0.1% to 90% by weight, from 1% to 80% by weight, or from 5% to 70% by weight, relative to the total weight of the composition.

### Pigments

In some embodiments, the composition is a keratin fiber dye composition that comprises pigments. Such a composition affords coloring remanent coatings, without degrading the keratin fibers. In other embodiments, the composition does not contain pigments. The present disclosure provides processes of treating keratinous fibers with compositions either with or without pigments.

The term “pigments” means any pigment that gives color to keratin materials. Their solubility in water at 25° C. and at atmospheric pressure (760 mmHg) is less than 0.05%; in some embodiments, their solubility in water at 25° C. and at atmospheric pressure (760 mmHg) is less than 0.01%.

The pigments may be chosen from the organic and/or mineral pigments known in the art, such as, for example, those described in Kirk-Othmer's Encyclopaedia of Chemical Technology and in Ullmann's Encyclopaedia of Industrial Chemistry.

These pigments may be in the form of pigmentary powder or paste. They may be coated or uncoated.

The pigments may be chosen, for example, from mineral pigments, organic pigments, lakes, pigments with special effects such as nacres or glitter flakes, and mixtures thereof.

The pigment may be chosen from natural pigments. The term “natural pigment” means any pigment that satisfies the definition in Ullmann's encyclopaedia in the chapter on inorganic pigments. Natural pigments may be chosen from, for example, iron oxides, chromium oxides, manganese violet, ultramarine blue, titanium dioxide, chromium hydrate and ferric blue.

The pigment may be an organic pigment. The term “organic pigment” means any pigment that satisfies the definition in Ullmann's encyclopaedia in the chapter on organic pigments. The organic pigment may be chosen from nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

White or colored organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Examples that may also be mentioned include pigmentary pastes of organic pigments, such as the product sold by the company Hoechst under the names:

Jaune Cosmenyl IOG: Pigment Yellow 3 (CI 11710);

Jaune Cosmenyl G: Pigment Yellow 1 (CI 11680);

Orange Cosmenyl GR: Pigment Orange 43 (CI 71105);

Rouge Cosmenyl R: Pigment Red 4 (CI 12085);

Carmine Cosmenyl FB: Pigment Red 5 (CI 12490);

Violet Cosmenyl RL: Pigment Violet 23 (CI 51319);

Bleu Cosmenyl A2R: Pigment Blue 15.1 (CI 74160);

Vert Cosmenyl GG: Pigment Green 7 (CI 74260);

Noir Cosmenyl R: Pigment Black 7 (CI 77266).

The pigments may also be in the form of composite pigments as described in patent EP 1 184 426. These composite pigments may be compounds of particles comprising a mineral core, at least one binder for ensuring the binding of the organic pigments to the core, and at least one organic pigment at least partially covering the core.

The organic pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform colored appearance (characterized by a certain shade, a certain vivacity and a certain lightness) that changes as a function of the conditions of observation (light, temperature, observation angles, etc.). They thus contrast with white or colored pigments that afford a standard uniform opaque, semi-transparent or transparent shade.

Several types of pigment with special effects exist: those with a low refractive index, such as fluorescent, photochromic or thermochromic pigments, and those with a high refractive index, such as nacres or glitter flakes.

Examples of pigments with special effects that may be mentioned include nacreous pigments such as white nacreous pigments such as mica coated with titanium or with bismuth oxychloride, colored nacreous pigments such as titanium mica with iron oxides, titanium mica with ferric blue or with chromium oxide, titanium mica with an organic pigment of the abovementioned type, and also nacreous pigments based on bismuth oxychloride. Nacreous pigments that may be mentioned include the Cellini nacres sold by Engelhard (mica-TiO₂-lake), Prestige sold by Eckart (mica-TiO₂), Prestige Bronze sold by Eckart (mica-Fe₂O₃), and Colorona sold by Merck (mica-TiO₂—Fe₂O₃).

In addition to nacres on a mica support, multilayer pigments based on synthetic substrates such as alumina, silica, sodium calcium borosilicate or calcium aluminium borosilicate, and aluminium, may be envisaged.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that may be used makes it possible to obtain a wide range of colors, and also particular optical effects such as metallic effects or interference effects.

The size of the pigments may range from 10 nm to 200 μm, from 20 nm to 80 μm, or from 30 nm to 50 μm.

The pigments may be dispersed in the product by means of a dispersant.

The dispersant serves to protect the dispersed particles against agglomeration or flocculation. This dispersant may be a surfactant, an oligomer, a polymer or a mixture of several thereof, bearing one or more functionalities with strong affinity for the surface of the particles to be dispersed. For example, they can physically or chemically attach to the surface of the pigments. These dispersants also contain at least one functional group that is compatible with or soluble in the continuous medium. For example, 12-hydroxystearic acid esters and C₈ to C₂₀ fatty acid esters of polyols such as glycerol or diglycerol are used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the cosmetic composition may be surface-treated with an organic agent.

Thus, the pigments that have been surface-treated beforehand are pigments that have totally or partially undergone a surface treatment of chemical, electronic, electrochemical, mechanochemical or mechanical nature, with an organic agent such as those described, for example, in Cosmetics and Toiletries, February 1990, Vol. 105, pp. 53-64, before being dispersed in the composition in accordance with the present disclosure. These organic agents may be chosen, for example, from amino acids; waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; fluorosilicone compounds.

The surface-treated pigments may also have been treated with a mixture of these compounds and/or may have undergone several surface treatments.

The surface-treated pigments may be prepared according to surface-treatment techniques that are well known to those skilled in the art, or may be commercially available in the required form.

The surface-treated pigments may be coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is described, for example, in U.S. Pat. No. 4,578,266.

An organic agent covalently bonded to the pigments may be used.

The agent for the surface treatment may represent from 0.1% to 50% by weight, from 0.5% to 30% by weight, or from 1% to 10% by weight relative to the total weight of the surface-treated pigments.

The surface treatments of the pigments may be chosen from the following treatments:

a PEG-silicone treatment, for instance the AQ surface treatment sold by LCW;

a chitosan treatment, for instance the CTS surface treatment sold by LCW;

a triethoxycaprylylsilane treatment, for instance the AS surface treatment sold by LCW;

a methicone treatment, for instance the SI surface treatment sold by LCW;

a dimethicone treatment, for instance the Covasil 3.05 surface treatment sold by LCW;

a dimethicone/trimethyl siloxysilicate treatment, for instance the Covasil 4.05 surface treatment sold by LCW;

a lauroyllysine treatment, for instance the LL surface treatment sold by LCW;

a lauroyllysine dimethicone treatment, for instance the LL/SI surface treatment sold by LCW;

a magnesium myristate treatment, for instance the MM surface treatment sold by LCW;

an aluminium dimyristate treatment, for instance the MI surface treatment sold by Miyoshi;

a perfluoropolymethylisopropyl ether treatment, for instance the FHC surface treatment sold by LCW;

an isostearyl sebacate treatment, for instance the HS surface treatment sold by Miyoshi;

a disodium stearoyl glutamate treatment, for instance the NAI surface treatment sold by Miyoshi;

a dimethicone/disodium stearoyl glutamate treatment, for instance the SA/NAI surface treatment sold by Miyoshi;

a perfluoroalkyl phosphate treatment, for instance the PF surface treatment sold by Daito;

an acrylate/dimethicone copolymer and perfluoroalkyl phosphate treatment, for instance the FSA treatment sold by Daito;

a polymethylhydrogenosiloxane/perfluoroalkyl phosphate treatment, for instance the FS01 surface treatment sold by Daito;

a lauroyllysine/aluminium tristearate treatment, for instance the LL-AlSt surface treatment sold by Daito;

an octyltriethylsilane treatment, for instance the OTS surface treatment sold by Daito;

an octyltriethylsilane/perfluoroalkyl phosphate treatment, for instance the FOTS surface treatment sold by Daito;

an acrylate/dimethicone copolymer treatment, for instance the ASC surface treatment sold by Daito;

an isopropyl titanium triisostearate treatment, for instance the ITT surface treatment sold by Daito;

a microcrystalline cellulose and carboxymethylcellulose treatment, for instance the AC surface treatment sold by Daito;

a cellulose treatment, for instance the C2 surface treatment sold by Daito;

an acrylate copolymer treatment, for instance the APD surface treatment sold by Daito;

a perfluoroalkyl phosphate/isopropyl titanium triisostearate treatment, for instance the PF+ITT surface treatment sold by Daito.

The composition may comprise one or more surface-untreated pigments.

When they are present, the amount of pigments may range from 0.1% to 40% by weight or from 0.5% to 20% by weight of the total weight of the composition.

### Additional Compounds

The composition may contain colored or coloring species such as hydrophilic or hydrophobic direct dyes or dye precursors.

The composition may contain particular silicone compounds other than those described previously, such as, for example, dimethiconols such as DC1501 Fluid.

The composition may contain a grafted silicone polymer. The term “grafted silicone polymer” as used herein means a polymer comprising a polysiloxane portion and a portion constituted by a non-silicone organic chain, one of the two portions constituting the main chain of the polymer, the other being grafted onto the said main chain.

The grafted silicone polymers used in the composition may be chosen from the group constituted by polymers with a non-silicone organic backbone grafted with monomers containing a polysiloxane, polymers with a polysiloxane backbone grafted with non-silicone organic monomers, and mixtures thereof.

The composition may also comprise a non-silicone polymer that can improve either the intrinsic properties of the composition, or the coating obtained during application to the hair, or both.

Such a polymer may be chosen from the following polymers:

polymers that are soluble in an organic liquid medium, in particular liposoluble polymers;

polymers that are dispersible in an organic solvent medium, such as, for example, polymers in the form of non-aqueous dispersions of polymer particles with a primary size of less than 1 μm, which may be dispersions in silicone oils or hydrocarbon-based oils;

polymers in the form of aqueous dispersions of polymer particles with a primary size of less than 1 μm, which are often referred to as “latices” (in this case, the composition comprises an aqueous phase);

water-soluble polymers (in this case, the composition comprises an aqueous phase or the polymer is applied as a pre-treatment or post-treatment to the composition).

The polymer that may be used in the composition may be anionic, cationic, nonionic or amphoteric.

The composition may also contain fillers, which are generally substantially uncolored compounds that are solid at room temperature and atmospheric pressure (760 mmHg), and insoluble in the composition, even when these ingredients are brought to a temperature above room temperature.

The fillers may be mineral or organic. The fillers may be particles of any shape, for example, platelet-shaped, spherical or oblong, irrespective of their crystallographic form (for example lamellar, cubic, hexagonal or orthorhombic). Furthermore, these particles may be solid, hollow or porous, and coated or uncoated.

Among the fillers that may be used in the compositions, mention may be made of mineral fillers such as talc, natural or synthetic mica; kaolin; boron nitride, precipitated calcium carbonate; magnesium carbonate, magnesium hydrogen carbonate; hydroxyapatite, cerium oxide, zirconium oxide.

The mineral particles may have a number-average primary size ranging from 0.1 to 30 μm, from 0.2 to 20 μm, or from 0.5 to 15 μm. The term “primary particle size” as used herein means the maximum dimension that it is possible to measure between two diametrically opposite points on an individual particle. The size of the organic particles may be determined by transmission electron microscopy or by measuring the specific surface area via the BET method, or via laser granulometry.

The mineral fillers may be, for example, talc, boron nitride or titanium dioxide.

Among the fillers that may be used, mention may be made of organic fillers. The term “organic filler” means a polymer particle that may be derived from the polymerization of one or more monomers. The polymers constituting these organic particles may be crosslinked or non-crosslinked. The monomers used may be, for example, esters of methacrylic or acrylic acid, such as methyl acrylate and methacrylate, vinylidene chloride, acrylonitrile, or styrene and derivatives thereof.

The organic particles may have a number-average primary size ranging from 1 to 30 μm, from 1 to 20 μm, or from 1 to 15 μm.

The organic particles used in the composition may be chosen from, for example, polyamide powders, acrylic polymer powders (including polymethyl methacrylate), acrylic copolymer powders (including of polymethyl methacrylate/ethylene glycol dimethacrylate), of polyallyl methacrylate/ethylene glycol dimethacrylate, of ethylene glycol dimethacrylate/lauryl methacrylate copolymer, or of polyacrylate/alkyl acrylate, polystyrene powders, and polyethylene powders (including of polyethylene/acrylic acid).

Non-limiting illustrations that may be mentioned as organic particles include:

polyamide (NYLON®) powders, for example those sold under the names ORGASOL® 4000 and ORGASOL® 2002 UD Nat Cos 204 by the company Atochem;

acrylic polymer powders (including of polymethyl methacrylate), for instance those sold under the name COVABEAD® LH85 and COVABEAD® PMMA by the company LCW or those sold under the name MICROPEARL® MHB sold by the company Matsumoto;

acrylic copolymer powders (including of polymethyl methacrylate/ethylene glycol dimethacrylate), for instance those sold under the name Dow Corning 5640 MICROSPONGE® Skin Oil Adsorber by the company Dow Corning, or those sold under the name GANZPEARL® GMP-0820 by the company Ganz Chemical, of polyallyl methacrylate/ethylene glycol dimethacrylate, for instance those sold under the name POLYPORE® L200 or POLYPORE® E200 sold by the company Amcol, of ethylene glycol dimethacrylate/lauryl methacrylate copolymer, for instance those sold under the name POLYTRAP® 6603 by the company Dow Corning, or of polyacrylate/ethylhexyl acrylate, for instance those sold under the name TECHPOLYMER® ACX 806C by the company Sekisui;

polystyrene/divinylbenzene powders, for instance those sold under the name TECHPOLYMER® SBX8 by the company Sekisui;

polyethylene powders, for example, of polyethylene/acrylic acid sold under the name FLOBEADS® by the company Sumitomo;

acrylic polymer microspheres such as those made of the crosslinked acrylate copolymer POLYTRAP 6603 ADSORBER® from the company RP Scherrer;

polyurethane powders such as the powder of the copolymer of hexamethylene diisocyanate and of trimethylol hexyl lactone sold under the name PLASTIC POWDER D-400® by the company Toshiki;

microcapsules of methyl acrylate or methacrylate polymers or copolymers, or

alternatively of copolymers of vinylidene chloride and of acrylonitrile, for instance EXPANCEL® from the company Expancel;

elastomeric crosslinked organopolysiloxane powders such as those sold under the name Trefil Powder E-506C by the company Dow Corning;

polyfluoro powders, for example, of polytetrafluoroethylene, for example the product sold under the name MP 1400 by the company Dupont de Nemours.

The organic particles used in the composition may be chosen from polyamide powders and polymethyl methacrylate powders.

The compositions may also comprise one or more oil thickeners chosen from polymeric thickeners and mineral thickeners, and mixtures thereof.

The polymeric thickener is, for example, an amorphous polymer formed by polymerization of an olefin. The olefin may be, for example, an elastomeric ethylenically unsaturated monomer.

Examples of olefins that may be mentioned include ethylenic carbide monomers, which may contain one or two ethylenic unsaturations, containing from 2 to 5 carbon atoms, such as ethylene, propylene, butadiene or isoprene.

The polymeric thickener is capable of thickening or gelling the organic phase of the composition. The term “amorphous polymer” means a polymer that does not have a crystalline form. The polymeric thickener may also be film-forming.

The polymeric thickener may be a diblock, triblock, multiblock, radial or star copolymer, or mixtures thereof.

Such polymeric thickeners are described in U.S. Pat. No. 5,221,534.

The polymeric thickener may be an amorphous block copolymer of styrene and of olefin.

The polymeric thickener may be hydrogenated to reduce the residual ethylenic unsaturations after polymerization of the monomers.

The polymeric thickener may be an optionally hydrogenated copolymer, containing styrene blocks and ethylene/C₃-C₄ alkylene blocks.

Diblock copolymers, which may be hydrogenated, that may be mentioned include styrene-ethylene/propylene copolymers and styrene-ethylene/butadiene copolymers. Mention may be made of the diblock polymers sold under the name KRATON® G1701 E by the company Kraton Polymers.

Triblock copolymers, which may be hydrogenated, that may be mentioned include styrene-ethylene/propylene-styrene copolymers, styrene-ethylene/butadiene-styrene copolymers, styrene-isoprene-styrene copolymers and styrene-butadiene-styrene copolymers. Mention may be made of the triblock polymers sold under the names KRATON® G1650, KRATON® G1652, KRATON® D1101, KRATON® D1102 and KRATON® D1160 by the company Kraton Polymers.

A mixture of hydrogenated styrene-butadiene/ethylene-styrene triblock copolymer and of hydrogenated ethylene-propylene-styrene star polymer may also be used. Such a mixture may be in isododecane. Such mixtures are sold, for example, by the company Penreco under the trade names VERSAGEL® M5960 and VERSAGEL® M5670.

A diblock copolymer such as those described above, such as, for example, a styrene-ethylene/propylene diblock copolymer, may be used as polymeric thickener.

The polymeric thickener may be present in a content ranging from 0.1% to 10% by weight, from 0.5% to 8% by weight, or from 1% to 5% by weight relative to the total weight of the composition.

The composition may also comprise one or more mineral oil-thickening agents such as an organophilic clay or fumed silicas.

Organophilic clays are clays modified with chemical compounds that make the clay able to swell in oily media.

Clays are products that are already well known per se, which are described, for example, in the publication “Mineralogie des argiles [Mineralogy of clays], S. Caillère, S. Hénin, M. Rautureau, 2nd Edition 1982, Masson”, the teaching of which is included herein by way of reference.

Clays are silicates containing a cation that may be chosen from calcium, magnesium, aluminium, sodium, potassium and lithium cations, and mixtures thereof.

Examples of such products that may be mentioned include clays of the smectite family such as montmorillonites, hectorites, bentonites, beidellites and saponites, and also of the vermiculite, stevensite and chlorite families.

These clays may be of natural or synthetic origin, and may be cosmetically compatible and acceptable with keratin materials such as the skin.

The organophilic clay may be chosen from montmorillonite, bentonite, hectorite, attapulgite and sepiolite, and mixtures thereof. The clay may be a bentonite or a hectorite.

These clays may be modified with a chemical compound chosen from quaternary amines, tertiary amines, amine acetates, imidazolines, amine soaps, fatty sulfates, alkyl aryl sulfonates and amine oxides, and mixtures thereof.

Organophilic clays that may be mentioned include quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay.

The fumed silicas may be obtained by high-temperature hydrolysis of a volatile silicon compound in an oxhydric flame, producing a finely divided silica. This process makes it possible to obtain hydrophilic silicas having a large number of silanol groups at their surface. Such hydrophilic silicas are sold, for example, under the names AEROSIL 130®, AEROSIL 200®, AEROSIL 255®, AEROSIL 300® and AEROSIL 380® by the company Degussa, and CAB-O-SIL HS-5®, CAB-O-SIL EH-5®, CAB-O-SIL LM-130®, CAB-O-SIL MS-55® and CAB-O-SIL M-5® by the company Cabot.

It is possible to chemically modify the surface of the said silica, via a chemical reaction generating a reduction in the number of silanol groups. It is possible to substitute silanol groups with hydrophobic groups: a hydrophobic silica is then obtained.

The hydrophobic groups may be:

trimethylsiloxyl groups, which may be obtained by treating fumed silica in the presence of hexamethyldisilazane. Silicas thus treated are known as “silica silylate” according to the CTFA (6th Edition, 1995). They are sold, for example, under the references AEROSIL R812® by the company Degussa and CAB-O-SIL TS-530® by the company Cabot;

dimethylsilyloxyl or polydimethylsiloxane groups, which may be obtained by treating fumed silica in the presence of polydimethylsiloxane or dimethyldichlorosilane. Silicas thus treated are known as “silica dimethyl silylate” according to the CTFA (6th Edition, 1995). They are sold, for example, under the references AEROSIL R972® and AEROSIL R974® by the company Degussa and CAB-O-SIL TS-610® and CAB-O-SIL TS-720® by the company Cabot.

The fumed silica may have a particle size that may be nanometric to micrometric, for example ranging from about 5 to 200 nm.

An organomodified bentonite or hectorite may be used as mineral thickener.

The mineral oil-thickening agent may be present in the composition in a content ranging from 0.1% to 8% by weight, from 0.2% to 6% by weight, or from 0.5% to 4% by weight relative to the total weight of the composition.

The compositions may also contain at least one agent usually used in cosmetics, chosen, for example, from reducing agents, fatty substances, plasticizers, softeners, antifoams, moisturizers, UV-screening agents, mineral colloids, peptizers, solubilizers, fragrances, proteins, vitamins, propellants, oxyethylenated or non-oxyethylenated waxes, paraffins, C₁₀-C₃₀ fatty acids such as stearic acid or lauric acid, and C₁₀-C₃₀ fatty amides such as lauric diethanolamide.

The above additives may be present in an amount for each of them ranging from 0.01% to 20% by weight relative to the weight of the composition.

Needless to say, a person skilled in the art will take care to select this or these optional additives(s) such that the advantageous properties intrinsically associated with the formation of the coating are not, or are not substantially, adversely affected.

The composition may be in the form of a suspension, a dispersion, a solution, a gel, an emulsion, an oil-in-water (O/W) or water-in-oil (W/O) emulsion, or a multiple emulsion (W/OWN or polyol/O/W or O/W/O), in the form of a cream, a mousse, a stick, a dispersion of vesicles (of ionic or nonionic lipids), a two-phase or multiphase lotion, a spray, a powder or a paste.

The composition may be an anhydrous composition, i.e. a composition containing less than 2% by weight of water. In some embodiments, the composition may contain less than 0.5% water, the water not being added during preparation of the composition but corresponding to the residual water provided by the mixed ingredients. In some embodiments, the composition may be free of water. The compositions may also be in the form of a lacquer.

The composition may be used on dry or wet hair. The additives described previously, when they are present, may be applied to the hair simultaneously with the composition or separately. The composition may be rinsed out or left in. It is also possible subsequently to wash the hair, this washing not being obligatory.

An application process with heating may also be used. According to this particular mode, the application to the hair is performed, for example, using a comb, a fine brush, a coarse brush or the fingers.

The application of the composition may then be followed by drying at a temperature above 40° C. According to one at least one embodiment, this temperature is greater than 45° C. According to at least one other embodiment, this temperature ranges from 45° C. to 220° C.

Drying may be performed immediately after the application or after a leave-on time that may range from 1 minute to 30 minutes. In some embodiments, the leave-on time is followed by optionally rinsing and/or washing.

In some embodiments, the hair is dried, in addition to supplying heat, with a flow of air. This flow of air during drying makes it possible to improve the individualization of the coating.

During drying, a mechanical action on the locks may be exerted, such as combing, brushing or running the fingers through.

The drying step of the process may be performed with a hood, a hairdryer, a smoothing iron, a Climazon, etc.

When the drying step is performed with a hood or a hairdryer, the temperature from the hood or hairdryer may range from 40 to 110° C. or from 50 to 90° C.

When the drying step is performed with a smoothing iron, the drying temperature of the smoothing iron may range from 110 and 220° C. or from 140 to 200° C.

Once the drying is complete, a final rinse or shampoo wash may optionally be performed.

The disclosure will be illustrated more fully using the non-limiting examples that follow.

### EXAMPLES

### Example 1

The following composition was produced:

| | Composition |
|---|---|
| | A |
| Volatile cyclic silicone DC245 Fluid (*) | 81 g |
| Polymethylsilsesquioxane sold under the name Wacker | 2 g |
| Belsil PMS MK Powder by the company Wacker | |
| α,ω-Dihydroxyl | 7 g |
| polydimethylsiloxane/cyclopentadimethylsiloxane | |
| mixture (14.7/85.3) sold under the name DC1501 Fluid (*) | |
| BioPSA 7-4405 (BioPSA 7-4400 diluted to 40% in | 15 g |
| isododecane) (*) | |

(*) sold by Dow Corning

0.3 g of the composition was applied to a 1-g lock of clean, wet hair of tone depth 4. After a leave-on time of 15 minutes, the lock was dried with a hairdryer for 2 minutes. A lock whose hairs were individualized and coated was obtained. This coating was shampoo-remanent.

### Example 2

The following composition was prepared:

| | Composition |
|---|---|
| | B |
| Isododecane | 79 g |
| Polypropylsilsesquioxane diluted to 50% by weight | 4 g |
| in D5, sold under the name DC670 Fluid (*) | |
| Linear silicone DC200 Fluid 500 000 cSt (*) | 2 g |
| BioPSA 7-4405 (BioPSA 7-4400 diluted to 40% in | 15 g |
| isododecane) (*) | |

(*) sold by Dow Corning

0.3 g of the composition was applied to a 1-g lock of clean, wet hair of tone depth 4. After a leave-on time of 15 minutes, the lock was dried with a hairdryer for 2 minutes. A lock whose hairs were individualized and coated was obtained. This coating was shampoo-remanent.

### Example 3

The following compositions were prepared:

Composition

| | C1 | C2 |
|---|---|---|
| Volatile cyclic silicone DC245 Fluid (*) | 42 g | 45 g |
| α,ω-Dihydroxyl polydimethylsiloxane/cyclo- | 10 g | 10 g |
| pentadimethylsiloxane mixture (14.7/85.3) sold | | |
| under the name DC1501 Fluid (*) | | |
| Polymethylsilsesquioxane sold under the | 3 g | — |
| name Wacker Belsil PMS MK Powder by the | | |
| company Wacker | | |
| Mica nacre coated with brown iron oxide, | 10 g | 10 g |
| sold by Eckart under the name Prestige Bronze | | |
| Disteardimonium hectorite (10%) and | 15 g | 15 g |
| propylene carbonate (3%) in isododecane, sold by | | |
| Elementis under the name Bentone Gel ISD V | | |
| BioPSA 7-4405 (BioPSA 7-4400 diluted to | 20 g | 20 g |
| 40% in isododecane) (*) | | |

(*) sold by Dow Corning

For compositions C1 and C2, 0.5 g of the composition were each applied to a 1-g lock of clean, wet hair with a tone depth of 4. After a leave-on time of 15 minutes, the lock was dried with a hairdryer for 2 minutes. Each of the compositions produced a colored lock whose hairs were individualized and whose color was shampoo-remanent. However, the feel of the lock obtained with composition C1 was less tacky than the feel of the lock obtained with composition C2.

### Example 4

The following composition was prepared:

| | Composition |
|---|---|
| | D |
| Volatile cyclic silicone DC245 Fluid (*) | 39 g |
| α,ω-Dihydroxyl polydimethylsiloxane/cyclo- | 10 g |
| pentadimethylsiloxane mixture (14.7/85.3) sold under the | |
| name DC1501 Fluid (*) | |
| Polypropylsilsesquioxane diluted to 50% by weight in | 6 g |
| D5, sold under the name DC670 Fluid (*) | |
| Mica nacre coated with brown iron oxide, sold by Eckart | 10 g |
| under the name Prestige Bronze | |
| Disteardimonium hectorite (10%) and propylene | 15 g |
| carbonate (3%) in isododecane, sold by Elementis under the | |
| name Bentone Gel ISD V | |
| BioPSA 7-4405 (BioPSA 7-4400 diluted to 40% in | 20 g |
| isododecane) (*) | |

(*) sold by Dow Corning

0.5 g of the composition was applied to a 1-g lock of clean, wet hair of tone depth 4. After a leave-on time of 15 minutes, the lock was dried with a hairdryer for 2 minutes. A colored lock whose hairs were individualized and whose color was shampoo-remanent was obtained

### Example 5

The following composition was prepared:

| | Composition |
|---|---|
| | E |
| Isododecane | 50 g |
| Linear silicone DC200 Fluid 500 000 cSt (*) | 2 g |
| Polymethylsilsesquioxane sold under the name Wacker | 3 g |
| Belsil PMS MK Powder by the company Wacker | |
| Mica nacre coated with brown iron oxide, sold by Eckart | 10 g |
| under the name Prestige Bronze | |
| Disteardimonium hectorite (10%) and propylene | 15 g |
| carbonate (3%) in isododecane, sold by Elementis under the | |
| name Bentone Gel ISD V | |
| BioPSA 7-4405 (BioPSA 7-4400 diluted to 40% in | 20 g |
| isododecane) (*) | |

(*) sold by Dow Corning

0.5 g of the composition was applied to a 1-g lock of clean, wet hair of tone depth 4. After a leave-on time of 15 minutes, the lock was dried with a hairdryer for 2 minutes. A colored lock whose hairs were individualized and whose color was shampoo-remanent was obtained.

What is claimed is:

1. A composition for treating keratin fibers, comprising
at least one copolymer comprising at least one silicone resin and at least one fluid silicone;
at least one volatile solvent; and
at least one silicone resin comprising at least one trifunctional unit of formula (R)SiO_{3/2}, wherein R is chosen, independently by trifunctional unit, from hydrocarbon-based groups and hydroxyl groups, on the condition that at least one R is a hydrocarbon-based group.

2. The composition according to claim 1, in which the at least one copolymer comprises the at least one silicone resin in a total content ranging from 45% to 75% and the at least one fluid silicone in a total content ranging from 25% to 55%, the sum of the percentages of the at least one silicone resin and of the at least one fluid silicone being equal to 100.

3. The composition according to claim 1, in which the at least one copolymer comprises the at least one silicone resin in a total content ranging from 55% to 65% and the at least one fluid silicone in a total content ranging from 35% to 45%, the sum of the percentages of the at least one silicone resin and of the at least one fluid silicone being equal to 100.

4. The composition according to claim 1, in which the at least one copolymer is present in a total amount greater than 1% by weight of the total weight of the composition.

5. The composition according to claim 1, in which the at least one volatile solvent is chosen from non-silicone organic solvents and silicone organic solvents.

6. The composition according to claim 1, in which the at least one volatile solvent is chosen from ethanol, isopropanol, acetone, alkanes that are liquid at 25° C. and at atmospheric pressure (760 mmHg), decamethylcyclopentasiloxane, octamethyltrisiloxane, decamethyltetrasiloxane, and dodecamethylcyclohexasiloxane.

7. The composition according to claim 6, in which the alkane that is liquid at 25° C. and at atmospheric pressure (760 mmHg) is isododecane.

8. The composition according to claim 1, in which the at least one silicone resin comprising at least one trifunctional unit of formula (R)SiO_{3/2} is chosen from the silsesquioxanes of formula: ((R′)SiO_{3/2})ₓ, in which x ranges from 100 to 500 and R′ is chosen, independently by trifunctional unit, from a hydrocarbon-based group containing from 1 to 10 carbon atoms or a hydroxyl group, on the condition that at least one R′ is a hydrocarbon-based group.

9. The composition according to claim 8, wherein the hydrocarbon-based group containing from 1 to 10 carbon atoms is a methyl group.

10. The composition according to claim 1, in which the molecular weight of the silicone resin ranges from 500 to 50,000 g/mol.

11. The composition according to claim 1, in which the at least one silicone resin comprising at least one trifunctional unit of formula (R)SiO_{3/2} is chosen from polymethylsilsesquioxanes, which are polysilsesquioxanes in which R is a methyl group; polypropylsilsesquioxanes, which are polysilsesquioxanes in which R is a propyl group; or polyphenylsilsesquioxanes, which are polysilsesquioxanes in which R is a phenyl group.

12. The composition according to claim 1, in which the weight ratio of (1) the at least one silicone resin comprising the at least one trifunctional unit of formula (R)SiO_{3/2}, to (2) the at least one volatile solvent ranges from 1/200 to 1/10.

13. The composition according to claim 1, further comprising at least one linear non-volatile polydimethylsiloxane with a viscosity of greater than 5 cSt at 25° C.

14. The composition according to claim 13, in which the at least one non-volatile linear polydimethylsiloxane has a viscosity ranging from 100 cSt at 25° C. to 4,000,000 cSt at 25° C.

15. The composition according to claim 13, wherein the at least one non-volatile linear polydimethylsiloxane with a viscosity of greater than 5 cSt at 25° C. is chosen from
polydimethylsiloxanes;
alkyl dimethicones;
polyphenylmethylsiloxanes; and
silicones modified with at least one group chosen from aliphatic groups, aromatic groups, fluorinated aliphatic groups, fluorinated aromatic groups, hydroxyl groups, thiol groups, and amine groups.

16. The composition according to claim 1, further comprising at least one pigment.

17. The composition according to claim 16, in which the at least one pigment is chosen from natural pigments.

18. The composition according to claim 17, in which the natural pigments are chosen from iron oxide, chromium oxide, manganese violet, ultramarine blue, titanium dioxide, chromium hydrate and ferric blue.

19. The composition according to claim 16, in which the at least one pigment is chosen from nacres.

20. The composition according to claim 1, in which the composition is anhydrous.

21. A process for treating keratin fibers comprising
applying to the keratin fibers a composition comprising
at least one copolymer comprising at least one silicone resin and at least one fluid silicone;
at least one volatile solvent; and
at least one silicone resin comprising at least one trifunctional unit of formula (R)SiO_{3/2}, wherein R is chosen, independently by trifunctional unit, from hydrocarbon-based groups and hydroxyl groups, on the condition that at least one R is a hydrocarbon-based group; and
optionally rinsing and/or washing.

22. The process of claim 21, in which, further comprising drying the keratin fibers by heating at a temperature greater than 40° C.

23. A process for obtaining a colored coating on hair comprising
applying to the keratin fibers a composition comprising
at least one copolymer comprising at least one silicone resin and at least one fluid silicone;
at least one volatile solvent; and
at least one silicone resin comprising at least one trifunctional unit of formula (R)SiO_{3/2}, wherein R is chosen, independently by trifunctional unit, from hydrocarbon-based groups and hydroxyl groups, on the condition that at least one R is a hydrocarbon-based group; and
at least one pigment.

24. A method for making a composition for treating hair comprising combining
at least one copolymer comprising at least one copolymer comprising at least one silicone resin and at least one fluid silicone;
at least one volatile solvent; and
at least one silicone resin comprising at least one trifunctional unit of formula (R)SiO_{3/2}, wherein R is chosen, independently by trifunctional unit, from hydrocarbon-based groups and hydroxyl groups, on the condition that at least one R is a hydrocarbon-based group.
